(19) United States
(12) Patent Application Publication
(10) Pub. No.: US 2003/0195373 A1
(43) Pub. Date: Oct. 16, 2003

(54) Novel beta-hydroxyamides

(76) Inventors: Mircea Manea, Vittsjo (SE); Cecilia Petersson, Helsingborg (SE)

Correspondence Address: Stevens Davis Miller & Mosher, Suite 850, 1615 L Street N W, Washington, DC, 20036, US

(21) Appl. No.: 10/311,295

(22) PCT Filed: Jun. 15, 2001
(86) PCT No.: PCT/SE01/01359

(30) Foreign Application Priority Data
Jun. 19, 2000 (SE) 0002268-1

(51) Int. Cl. C07C 233/45

(52) U.S. Cl. 560/41; 560/155

(57) ABSTRACT

Disclosed is a noble β-hydroxyamide of general formula wherein R¹ is alkyl, alkoxyalkyl or hydroxyalkoxyalkyl derived from at least one hydroxyfunctional compound, R² is alkyl, aryl, alkylaryl or arylalkyl derived from at least one carboxyfunctional compound or at least one anhydride, halide or ester of at least one carboxyfunctional compound, R³ is N-alkyl or N-cycloalkyl derived from at least one alkanolamine and wherein m and n are independent integers and at least 1. In a further aspect the present refers to a process for synthesis of said β-hydroxyamide. The process comprises the Steps of (i) subjecting a di, tri or polyalcohol to alcoholysis with at least one di, tri or polyalkyl ester of a di, tri or polyfunctional carboxylic acid and (ii) subjecting obtained reaction product to aminolysis with at least one alkanolamine.

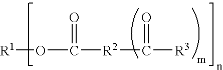

The present invention refers to a novel series of β-hydroxyamides, such as β-hydroxyalkylamides, β-hydroxyarylalkylamides and β-hydroxyalkylarylamides, having a core being derived from a di, tri or polyhydric compound, to which core at least one alkylamide, arylalkylamide or alkylarylamide core branch is bonded. Said β-hydroxyamides are useful as chemical intermediates and chemical crosslinkers and/or curing agents. In a further aspect, the present invention refers to a process for synthesis of said β-hydroxyamides, which process comprises the Steps of (i) alcoholysis of said di, tri or polyhydric compound and a di, tri or poly(alkyl) ester and (ii) aminolysis of obtained reaction product with an alkanolamine.

β-hydroxyamides are well known in coating applications such as powder coatings as a challenging alternative to compounds such as triglycidyl trisisocyanurate (TGIC). β-hydroxyamides are normally produced by aminolysis of alkyl esters, such as diethyl esters of dicarboxylic acids, by β-aminoalcohols. β-hydroxyamides are normally solid and used for instance in powder coating compositions as crosslinkers and/or curing agents. Available patent literature discloses a number of processes for production of β-hydroxyamides, β-hydroxyamides yielded in said processes and various application areas for said β-hydroxyamides. European Patent Application 0 473 380, European Patent Application 0 960 878, U.S. Pat. No. 4,076,917, U.S. Pat. No. 4,727,111 and U.S. Pat. No. 5,101,073 disclose β-hydroxyalkylamides and processes for production of β-hydroxyalkylamides. Disclosed products are reaction products of a di, tri or polyfunctional carboxylic acid and at least one β-aminoalcohol. U.S. Pat. No. 2,703,798 and International Patent Applications WO 92/06070, WO 92/06072 and WO 92/06073 disclose processes yielding reaction products of aliphatic fatty acids and N-alkylglucamines. German Patent Applications 31 50 269 and 31 24 885 relate to poly-N,N-hydroxyalkylamides of di, tri or polyfunctional aromatic or cycloaliphatic carboxylic acids.

In spite of a strong expansion in powder coatings, there are still segments wherein the use of liquid coatings, presently for instance comprising saturated polyesters or alkyds combined with alkyl etherified urea-formaldehyde and/or melamine-formaldehyde resins as crosslinkers, is required. Various coating systems, binders, solvents and crosslinkers are thoroughly disclosed and discussed in readily available handbooks, such as “A Manual for Resins for Surface Coatings” Vol. I-III, G. Hayward, P. K. T. Oldring and C. J. S. Standen, SITA Technology, London, 1993-94, “Surface Coatings—Science & Technology”, S. Paul (ed.), John Wiley & Sons, 1996 and “Surface Coatings” vol. 1 “Raw Materials and Their Usage” and vol. 2 “Paints and Their Application”, Chapman & Hall Ltd, London, 1974 and 1984. Environmental issues regarding for instance the minimising of organic solvents and formaldehyde emission reduce the formulation possibilities when high performance is required or requested. The coil coating sector and other application areas wherein waterborne systems do not perform as well as solvent borne systems, and where powder coatings not are performing adequately require the presence of a suitable crosslinker complying with environmental issues as well as demands on high performance.

It has quite unexpectedly been found that β-hydroxyamides having a core derived from a di, tri or polyfunctional alcohol give the following advantages over presently known and available crosslinkers, including β-hydroxyamides being the reaction products of di, tri or polyfunctional carboxylic acids and aminoalkohols,

coatings can be formulated as a solvent borne or waterborne system,

solvent borne coatings can be formulated as high-solids systems,

crosslinking temperature may be moderate to high in the range of 150-200° C.,

compatibility problems are due to the di, tri or polyhydric core compound reduced,

the relation between flexibility and hardness is improved compared to formulations comprising amino-formaldehyde resins as crosslinkers, and

the crosslinking density can be tailored by a proper selection of the di, tri or polyhydric core compound.

The β-hydroxyamide of the present invention is a compound of general formula

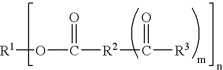

wherein

R¹ is alkyl, alkoxyalkyl, hydroxyalkyl or hydroxyalkoxyalkyl, R¹ being derived from at least one compound having at least two hydroxyl groups,

R² is alkyl, aryl, alkylaryl or arylalkyl, R² derived from at least one compound having at least two carboxyl groups or from an anhydride, a halide or an alkyl ester or ether of a compound having said at least two carboxyl groups,

R³ is N-alkyl or N-cycloalkyl having at least one hydroxyl group in β-position, R³ being derived from at least one alkanolamine,

m and n are independent integers, each being at least 1.

Substituent R¹ is preferably alkyl or alkoxyalkyl comprising at least two ester and/or ether groups and substituent R³ is likewise preferably a group of formula

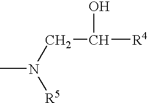

wherein R⁴ is alkyl and R⁵ is hydrogen or a group of formula

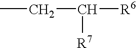

wherein R⁶ is alkyl and R⁷ is hydrogen, hydroxyl, alkyl or hydroxyalkyl.

The series of β-hydroxyamides of the present invention include embodiments wherein

alkyl is linear or branched alkanyl having 1-24 carbon atoms or linear or branched alkenyl having 2-24 carbon atoms, N-alkyl is N-alkanyl having 2-20 carbon atoms,

N-cycloalkyl is N-cycloalkanyl having 3-20 carbon atoms,

alkoxy in alkoxyalkyl is —(CᵣH₂ᵣO)ₚ— wherein r and p are independent integers being at least 1, and

hydroxyalkoxy in hydroxyalkoxyalkyl is —(CᵣH₂ᵣO)ₚH wherein r and p are independent integers being at least 1.

Alkoxy and/or hydroxyalkoxy above is/are preferably derived from at least one alkylene oxide, such as ethylene oxide, propylene oxide and/or butylene oxide.

The compound having said at least two hydroxyl groups is in preferred embodiments of the present invention a di, tri or polyalcohol of formula

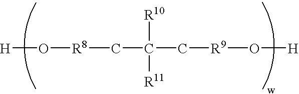

wherein

w is an integer and at least 1,

R⁸ is a group of formula (H₂ₓ₊₁Cₓ)_{y}—, (Oₚ₋₁H₂ᵣCᵣ)ₚ— or (Oₚ₋₁H₂ᵣCᵣ)ₚ(H₂ₓ₊₁Cₓ)_{y}— wherein x, y, r and p are independent intergers being at least 1,

R⁹ is a group of formula —(CₓH₂ₓ₊₁)_{y}, —(CᵣH₂ᵣOₚ₋₁)ₚ or —(CₓH₂ₓ₊₁)_{y}(CᵣH₂ᵣOₚ₋₁)ₚ wherein x, y, r and p are independent intergers being at least 1, and

R¹⁰ and R¹¹ is independently —H, —OH or a group of formula —(CₓH₂ₓ₊₁)_{y}, —(CₓH₂ₓ)_{y}OH, —(CᵣH₂ᵣO)ₚH, —(CₓH₂ₓ₊₁)_{y}(CᵣH₂ᵣO)ₚH, wherein x, y, r and p are independent intergers being at least 1.

Substituent R² is preferably derived from a di, tri or polyfunctional carboxylic acid, from an anhydride of a di, tri or polyfunctional carboxylic acid or from a di, tri or polyalkyl ester of a di, tri or polyfunctional carboxylic acid. Alkyl is for instance and preferably C₁-C₁₈, such as C₁-C₈ or C₁-C₄, linear or branched alkanyl.

The compound having said at least two hydroxyl groups is advantageously a di, tri or polyalcohol selected from the group consisting of a 2-alkyl-1,3-propanediol, a 2,2-dialkyl-1,3-propanediol, a 2-hydroxy-1,3-propanediol, a 2,2-dihydroxy-1,3-propanediol, a 2-hydroxy-2-alkyl-1,3-propanediol, a 2-hydroxyalkyl-2-alkyl-1,3-propanediol, a 2,2-di(hydroxyalkyl)-1,3-propanediol, a 2-hydroxyalkoxy-2-alkyl-1,3-propanediol, a 2,2-di(hydroxyalkoxy)-1,3-propanediol, a 2-hydroxyalkoxyalkyl-2-alkyl-1,3-propanediol, a 2,2-di(hydroxyalkoxy-alkyl)-1,3-propanediol and a dimer, trimer or polymer of a said 1,3-propanediol. Alkyl is in these embodiments preferably linear or branched alkanyl having 1-18 carbon atoms or linear or branched alkenyl having 3-18 carbon atoms and alkoxy and hydroxyalkoxy is for example derived from at least one alkylene oxide, such as ethylene oxide, propylene oxide and/or butylene oxide. The di, tri or polyalcohol can suitably be exemplified by compounds such as 2-methyl-1,3-propanediol, 2-methyl-2-ethyl-1,3-propanediol, 2-ethyl-2-butyl-1,3-propanediol, neopentyl glycol, dimethylpropane, trimethylolethane, trimethylolpropane, di(trimethylolethane), di(trimethylolpropane), pentaerythritol and dipentaerythritol.

Further suitable embodiments of the compound having said at least two hydroxyl groups are found among alcohols such as glycerol, diglycerol, anhydroenneaheptitol, sorbitol and mannitol as well as monoallyl or mono(methallyl) ethers of glycerol, trimethylolethane, trimethylolpropane, di(trimethylolethane), di(trimethylolpropane) and pentaerythritol and diallyl or di(methallyl) ethers of di(trimethylolethane), di(trimethylolpropane) or pentaerythritol.

Yet further suitable embodiments of the compound having said at least two hydroxyl groups include di, tri or poly(hydroxy)carboxylic acids, wherein the carboxyl group or groups optionally is/are protected using for instance well known protection methods as disclosed in handbooks such as “Protective Groups in Organic Synthesis” chapter 5 “Protection for the Carboxyl Group” by Theodora W. Green and Peter G. M. Wuts, John Wiley & Sons Inc., 1991. Said hydroxycarboxylic acids are most preferably selected from the group consisting of 2,2-dimethylolpropionic acid, α,α-bis(hydroxymethyl)butyric acid, α,α,α-tris(hydroxymethyl)acetic acid, α,α-bis(hydroxymethyl)valeric acid, α,α-bis(hydroxy)propionic acid, 3,5-di(hydroxy)benzoic acid, α,β-di(hydroxy)propionic acid, heptonic acid, citric acid, tartaric acid, di(hydroxy)maloic acid and gluconic acid.

The compound having said at least two carboxyl groups is most preferably a di, tri or polyfunctional carboxylic acid, which in the most preferred embodiments is selected from the group consisting of adipic acid, azelaic acid, fumaric acid, maleic acid, phthalic acid, isophthalic acid, terephthalic acid, tetrahydrophthalic acid, hexahydrophthalic acid, succinic acid, sebacic acid, diglycolic acid, trimelletic acid, citric acid and pyromelletic acid, or is an anhydride, a halide or an alkyl ester or ether of a said di, tri or polyfunctional carboxylic acid.

The alkanolamine as disclosed above is advantageously selected from the group consisting of monoethanolamine, diethanolamine, mono-n-propanolamine, di-n-propanolamine, monoisopropanolamine, diisopropanolamine, mono-n-butanolamine, di-n-butanolamine, monoisobutanolamine, diisobutanolamine, mono-sec-butanolamine, di-sec-butanolamine, methylethanolamine, n-butylethanolamine, isobutylethanolamine, N-acetylethanolamine, 2-aminocyclohexanol, 2-aminocyclopentanol, 2-amino-2-methyl-1,3-propanediol, 2-amino-2-ethyl-1,3-propanediol and 2-amino-2-hydroxymethyl-1,3-propanediol.

In a further aspect, the present invention refers to a process for synthesis of a β-hydroxyamide as disclosed above. The process comprises the steps of

(i) subjecting a di, tri or polyalcohol of formula

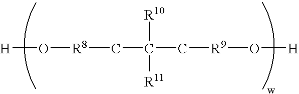

wherein

w is an integer and at least 1,

R⁸ is a group of formula (H₂ₓ₊₁Cₓ)_{y}—, (Oₚ₋₁H₂ᵣCᵣ)ₚ— or (Oₚ₋₁H₂ᵣCᵣ)ₚ(H₂ₓ₊₁Cₓ)_{y}— wherein x, y, r and p are independent integers being at least 1,

R⁹ is a group of formula —(CₓH₂ₓ₊₁)_{y}, —(CᵣH₂ᵣOₚ₋₁)ₚ or —(CₓH₂ₓ₊₁)_{y}(CᵣH₂ᵣOₚ₋₁)ₚ wherein x, y, r and p are independent integers being at least 1, and

R¹⁰ and R¹¹ each independently is —H, —OH, —COOH or a group of formula —(CₓH₂ₓ₊₁)_{y}, —(CₓH₂ₓ)_{y}OH, —(CₓH₂ₓ)_{y}COOH, —(CᵣH₂ᵣO)ₚH, —(CₓH₂ₓ₊₁)_{y}(CᵣH₂ᵣO)ₚH, wherein x, y, r

and p are independent integers being at least 1,

to alcoholysis with a di, tri or polyalkyl ester of a di, tri or polyfunctional carboxylic acid, said di, tri or polyalkyl ester having a formula of

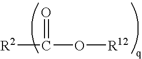

wherein

R² is alkyl, aryl, alkylaryl or arylalkyl, wherein alkyl is linear or branched alkanyl having 1-24 carbon atoms or linear or branched alkenyl having 2-24 carbon atoms,

R¹² is C₁-C₈ alkyl, preferably methyl, ethyl, propyl or butyl, and q is an interger and at least 2,

while removing by-product R¹²OH, and

(ii) subjecting the reaction product obtained in Step (i) to aminolysis with at least one alkanolamine while removing by-product R¹²OH.

Suitable reaction temperatures are for the alcoholysis as well as the aminolysis normally but not exclusively found within the range of 150-250° C., such as 160-220° C.

Preferred embodiments of the process according to the present invention include subjecting a di, tri or polyalcohol selected from the group consisting of 2-alkyl-1,3-propanediol, a 2,2-dialkyl-1,3-propanediol, a 2-hydroxy-1,3-propanediol, a 2,2-dihydroxy-1,3-propanediol, a 2-hydroxy-2-alkyl-1 ,3-propanediol, a 2-hydroxyalkyl-2-alkyl-1,3-propanediol, a 2,2-di(hydroxyalkyl)-1,3-propanediol, a 2-hydroxyalkoxy-2-alkyl-1,3-propanediol, a 2,2-di(hydroxyalkoxy)-1,3-propanediol, a 2-hydroxyalkoxyalkyl-2-alkyl-1,3-propanediol, a 2,2-di(hydroxyalkoxyalkyl)-1,3-propanediol and a dimer, trimer or polymer of a said 1,3-propanediol, to said alcoholysis with a di, tri or polyalkyl ester of a di, tri or polyfunctional carboxylic acid selected from the group consisting of adipic acid, azelaic acid, fumaric acid, maleic acid, phthalic acid, isophthalic acid, terephthalic acid, tetrahydrophthalic acid, hexahydrophthalic acid, succinic acid, sebacic acid, diglycolic acid, trimelletic acid, citric acid and pyromelletic acid. Alkyl is here suitably linear or branched alkanyl having 1-18 carbon atoms or linear or branched alkenyl having 3-18 carbon atoms and alkoxy and hydroxyalkoxy is derived from at least one alkylene oxide, such as ethylene oxide, propylene oxide and/or butylene oxide. Said di, tri or polyalcohol is suitably exemplified by compounds such as 2-methyl-1,3-propanediol, 2-methyl-2-ethyl-1,3-propanediol, 2-ethyl-2-butyl-1,3-propanediol, neopentyl glycol, dimethylpropane, trimethylolethane, trimethylolpropane, di(trimethylolethane), di(trimethylolpropane), pentaerythritol or dipentaerythritol.

Further suitable embodiments of the di, tri or polyalcohol include for instance di, tri or poly(hydroxy)carboxylic acid, wherein the carboxyl group or groups is/are protected using methods as previously disclosed. The most preferred embodiments of di, tri and poly(hydroxy)carboxylic acid include 2,2-dimethylolpropionic acid, α,α-bis(hydroxymethyl)butyric acid, α,α,α-tris(hydroxymethyl)acetic acid, α,α-bis(hydroxymethyl)valeric acid, α,α-bis(hydroxy)propionic acid, 3,5-di(hydroxy)benzoic acid, α,β-di(hydroxy)propionic acid, heptonic acid, citric acid, tartaric acid, di(hydroxy)maloic acid and gluconic acid.

The alkanolamine of Step (ii) is in preferred embodiments of the process selected from the group consisting of monoethanolamine, diethanolamine, mono-n-propanolamine, di-n-propanolamine, monoisopropanolamine, diisopropanolamine, mono-n-butanolamine, di-n-butanolamine, monoisobutanolamine, diisobutanolamine, mono-sec-butanolamine, di-sec-butanolamine, methylethanolamine, n-butylethanolamine, isobutylethanolamine, N-acetylethanolamine, 2-aminocyclohexanol, 2-aminocyclopentanol, 2-amino-2-methyl-1,3-propanediol, 2-amino-2-ethyl-1,3-propanediol and 2-amino-2-hydroxymethyl-1,3-propanediol.

Without further elaboration, it is believed that one skilled in the art can, using the preceding description, utilise the present invention to its fullest extent. The following preferred specific embodiments are, therefore, to be construed as merely illustrative and not limitative of the remainder of the disclosure in any way whatsoever. It will be understood, of course, that the invention is not limited thereto since many modifications may be made, and it is, therefore, contemplated to cover by the appended claims any such modifications as fall within the true spirit and scope of the invention. In the following Examples 1-8 refer to preparation of embodiments of the β-hydroxyamide and to embodiments of the process of the present invention. Examples 9 and refer to preparation and evaluation of a coating composition comprising β-hydroxyamides according to embodiments of the present invention.

### EXAMPLE 1

Step (i): 765.6 g of dimethyladipate was charged in a reaction flask equipped with electrical heating, a Dean-Stark separator, a vertical cooler, mechanical stirrer and nitrogen inlet. 0.1 g dibutyltinlaureate was under stirring and nitrogen blanket added. The temperature was now raised to 165° C. and 136 g of pentaerythritol added in small portions maintaining the temperature and progressively distilling off formed methanol. The temperature increased to 220° C. and the alcoholysis was considered completed when 128 g methanol was collected. Vacuum was applied and excess of dimethyladipate was evaporated from formed pentaerythritol tetra(methyladipate). The temperature was subsequently decreased to 180° C.

Step (ii) 420 g of diethanolamine was charged at said 180° C. The temperature was subsequently raised to 220° C. and methanol removed from the reaction mixture. The aminolysis was considered completed when 128 g of methanol was collected and vacuum was applied to remove unreacted diethanolamine.

Obtained β-hydroxyamide (1000 g) had a hydroxyl value of 8 mequiv/g.

### EXAMPLE 2

Step (i): 800 g of Estasol (mixed dibasic ester comprising 21%-w/w of dimethyladipate, 59%-w/w of dimethylglutarate and 20%-w/w of dimethylsuccinate), 193 g of dipentaerythritol, 3 g dibutyltinoxide and 2 g of trisnonylphenylphosphite were charged in a reaction flask equipped with electrical heating, a Dean-Stark separator, a vertical cooler, mechanical stirrer and nitrogen inlet. The temperature was under stirring and nitrogen blanket raised to 170° C. and formed methanol progressively distilled off. The temperature was maintained at 170° C. and the alcoholysis was considered completed when 140 ml of methanol was collected. Vacuum was applied and excess of Estasol was evaporated from formed dipentaerythritol ester.

Step (ii): 200 g of the dipentaerythritol ester obtained in Step (i), 124 g of diethanolamine and 0.3 g dibutyltinoxide were charged in a reaction flask equipped as in Step (i). The temperature slowly raised to 220° C. and methanol removed from the reaction mixture. The aminolysis was considered completed when 37 ml of methanol was collected. Vacuum was now applied to remove unreacted diethanolamine and obtained β-hydroxyamide was recovered.

Obtained β-hydroxyamide had a hydroxyl value of 8.23 mequiv/g and a viscosity of 740 mPas at 100° C.

### EXAMPLE 3

Step (i): 500 g of Estasol (mixed dibasic ester comprising 21%-w/w of dimethyladipate, 59%-w/w of dimethylglutarate and 20%-w/w of dimethylsuccinate), 106.5 g of pentaerythritol, 1.8 g dibutyltinoxide and 1.2 g of trisnonylphenylphosphite were charged in a reaction flask equipped with electrical heating, a Dean-Stark separator, a vertical cooler, mechanical stirrer and nitrogen inlet. The temperature was under stirring and nitrogen blanket raised to 185° C. and formed methanol progressively distilled off. The temperature was allowed to decrease to 165° C. during the alcoholysis and the alcoholysis was considered completed when 100 ml of methanol was collected and unreacted Estasol was distilled off from formed pentaerythritol ester.

Step (ii): 200 g of the pentaerythritol ester obtained in Step (i), 130 g of diethanolamine and 0.3 g dibutyltinoxide were charged in a reaction flask equipped as in Step (i). The temperature slowly raised to 220° C. and methanol removed from the reaction mixture. The aminolysis was considered completed when 39 ml of methanol was collected. Vacuum was now applied to remove unreacted diethanolamine and obtained β-hydroxyamide was recovered.

Obtained β-hydroxyamide had a hydroxyl value of 8.52 mequiv/g and a viscosity of 660 mPas at 100° C.

### EXAMPLE 4

Step (i): 500 g of Estasol (mixed dibasic ester comprising 21%-w/w of dimethyladipate, 59%-w/w of dimethylglutarate and 20%-w/w of dimethylsuccinate), 163 g of neopentyl glycol, 2 g dibutyltinoxide and 1.3 g of trisnonylphenylphosphite were charged in a reaction flask equipped with electrical heating, a Dean-Stark separator, a vertical cooler, mechanical stirrer and nitrogen inlet. The temperature was under stirring and nitrogen blanket raised the melting point of neopentyl glycol and formed methanol progressively distilled off. The temperature was allowed to decrease 5-10° C. during the alcoholysis and the alcoholysis was considered completed when 100 ml of methanol was collected and unreacted Estasol was distilled off from formed neopenyl glycol ester.

Step (ii): 200 g of the neopentyl glycol ester obtained in Step (i), 117 g of diethanolamine and 0.3 g dibutyltinoxide were charged in a reaction flask equipped as in Step (i). The temperature slowly raised to 220° C. and methanol removed from the reaction mixture. The aminolysis was considered completed when 35 ml of methanol was collected. Vacuum was now applied to remove unreacted diethanolamine and obtained β-hydroxyamide was recovered.

Obtained β-hydroxyamide had a hydroxyl value of 7.91 mequiv/g and a viscosity of 260 mPas at 100° C.

### EXAMPLE 5

Step (i): 500 g of Estasol (mixed dibasic ester comprising 21%-w/w of dimethyladipate, 59%-w/w of dimethylglutarate and 20%-w/w of dimethylsuccinate), 210 g of dimethylolpropionic acid, 2.1 g dibutyltinoxide and 1.4 g of trisnonylphenylphosphite were charged in a reaction flask equipped with electrical heating, a Dean-Stark separator, a vertical cooler, mechanical stirrer and nitrogen inlet. The temperature was under stirring and nitrogen blanket raised all dimethylolpropionic acid was dissolved and formed methanol progressively distilled off. The temperature was allowed to decrease 5-10° C. during the alcoholysis and the alcoholysis was considered completed when 100 ml of methanol was collected and unreacted Estasol was distilled off from formed dimethylolpropionic acid ester.

Step (ii): 200 g of the dimethylolpropionic acid ester obtained in Step (i), 108 g of diethanolamine and 0.3 g dibutyltinoxide were charged in a reaction flask equipped as in Step (i). The temperature slowly raised to 220° C. and methanol removed from the reaction mixture. The aminolysis was considered completed when 33 ml of methanol was collected. Vacuum was now applied to remove unreacted diethanolamine and obtained β-hydroxyamide was recovered.

Obtained β-hydroxyamide had a hydroxyl value of 7.47 mequiv/g and a viscosity of 840 mPas at 100° C.

### EXAMPLE 6

Step (i): 500 g of Estasol (mixed dibasic ester comprising 21%-w/w of dimethyladipate, 59%-w/w of dimethylglutarate and 20%-w/w of dimethylsuccinate), 141 g of 2-methyl-1,3-propanediol, 2 g dibutyltinoxide and 1.3 g of trisnonylphenylphosphite were charged in a reaction flask equipped with electrical heating, a Dean-Stark separator, a vertical cooler, mechanical stirrer and nitrogen inlet. The temperature was under stirring and nitrogen blanket raised to 140° C. and formed methanol progressively distilled off. The temperature was further raised to 160° C. and allowed to decrease 5-10° C. during the alcoholysis. The alcoholysis was considered completed when 100 ml of methanol was collected and unreacted Estasol was distilled off from formed 2-methyl-1,3-propanediol ester.

Step (ii): 200 g of the 2-methyl-1,3propanediol ester obtained in Step (i), 122 g of diethanolamine and 0.3 g dibutyltinoxide were charged in a reaction flask equipped as in Step (i). The temperature slowly raised to 220° C. and methanol removed from the reaction mixture. The aminolysis was considered completed when 37 ml of methanol was collected. Vacuum was now applied to remove unreacted diethanolamine and obtained β-hydroxyamide was recovered.

Obtained β-hydroxyamide had a hydroxyl value of 8.13 mequiv/g and a viscosity of 457 mPas at 100° C.

### EXAMPLE 7

Step (i): 800 g of Estasol (mixed dibasic ester comprising 21%-w/w of dimethyladipate, 59%-w/w of dimethylglutarate and 20%-w/w of dimethylsuccinate), 309.5 g of di(trimethylolpropane), 3.3 g dibutyltinoxide and 2.2 g of trisnonylphenylphosphite were charged in a reaction flask equipped with electrical heating, a Dean-Starkseparator, a vertical cooler, mechanical stirrer and nitrogen inlet. The temperature was under stirring and nitrogen blanket raised until all di(trimethylolpropane) was dissolved and formed methanol progressively distilled off. The temperature was allowed to decrease 5-10° C. during the alcoholysis. The alcoholysis was considered completed when 160 ml of methanol was collected and unreacted Estasol was distilled off from formed di(trimethylolpropane) ester.

Step (ii): 200 g of the di(trimethylolpropane) ester obtained in Step (i), 111 g of diethanolamine and 0.3 g dibutyltinoxide were charged in a reaction flask equipped as in Step (i). The temperature slowly raised to 220° C. and methanol removed from the reaction mixture. The aminolysis was considered completed when 34 ml of methanol was collected. Vacuum was now applied to remove unreacted diethanolamine and obtained β-hydroxyamide was recovered.

Obtained β-hydroxyamide had a hydroxyl value of 7.62 mequiv/g and a viscosity of 428 mPas at 100° C.

### EXAMPLE 8

Step (i): 500 g of Estasol (mixed dibasic ester comprising 21%-w/w of dimethyladipate, 59%-w/w of dimethylglutarate and 20%-w/w of dimethylsuccinate), 140 g of trimethylolpropane and 2 g dibutyltinoxide were charged in a reaction flask equipped with electrical heating, a Dean-Stark separator, a vertical cooler, mechanical stirrer and nitrogen inlet. The temperature was under stirring and nitrogen blanket raised to 160° C. and formed methanol progressively distilled off. The temperature was further raised to 200° C. and allowed to decrease 5-10° C. during the alcoholysis. The alcoholysis was considered completed when 100 ml of methanol was collected and unreacted Estasol was distilled off from formed trimethylolpropane ester.

Step (ii): 200 g of the trimethylolpropane ester obtained in Step (i), 122 g of diethanolamine and 0.3 g dibutyltinoxide were charged in a reaction flask equipped as in Step (i). The temperature slowly raised to 220° C. and methanol removed from the reaction mixture. The aminolysis was considered completed when 37 ml of methanol was collected. Vacuum was now applied to remove unreacted diethanolamine and obtained β-hydroxyamide was recovered.

Obtained β-hydroxyamide had a hydroxyl value of 8.5 mequiv/g and a viscosity of 820 mPas at 100° C.

### EXAMPLE 9

Preparation and evaluation of a coating composition comprising a β-hydroxyamide according to an embodiment of the present invention.

A lacquer was prepared by mixing 1 g of the â-hydroxylamide obtained in Example 1 with a polyurethane dispersion having the following composition:

| | |
|---|---|
| N-methylpyrrolidone: | 7.37 g |
| isophorone diisocyanate: | 11.61 g |
| dimethylolpropionic acid: | 1.50 g |
| polypropylene glycol adipate: | 11.67 g |
| trimethylolpropane: | 0.14 g |
| dimethylethanolamine: | 1.30 g |
| ethylenediamine: | 1.02 g |
| water: | 56.00 g |

0.20 g of a silicon defoamer was and 0.50 g a dimethylsiloxane based substrate wetting agent was finally mixed into obtained lacquer composition. The lacquer, a milky waterborne liquid, was applied on a glass substrate at a thickness of 120 ìm wet film and cured at 150° C. to yield a clear coating having a König hardness of 100 oscillations.

### EXAMPLE 10

Preparation and evaluation of a coating composition comprising a β-hydroxyamide according to an embodiment of the present invention.

A polyester was prepared according to a standard procedure from 2 moles of endomethylene tetrahydrophthalic anhydride and 1 mole of pentaerythritol. Obtained polyester had an acid value of 95-110 mg KOH/g and was diluted to a non-volatile content of 75% by weight in xylene.

A lacquer was prepared by mixing 100 g said polyester with 21 g of the â-hydroxylamide obtained in Example 8. The lacquer was applied on a glass substrate at a thickness of 150 \grave{m} wet film and cured at 200° C. to yield a clear coating having a König hardness of 150 oscillations.

1. A novel β-hydroxyamide characterised in, that it has a general formula of 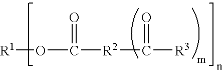
wherein
R¹ is alkyl or alkoxyalkyl, R² is alkyl, aryl, alkylaryl or arylalkyl, R³ is N-alkyl or N-cycloalkyl having at least one hydroxyl group in β-position, m is an integer and at least 1 and wherein n is an integer and at least 2.

2. A novel β-hydroxyamide according to claim 1 characterised in, that it is obtained by subjecting a di, tri or polyalcohol to alcoholysis with a di, tri or polyalkyl ester of a di, tri or polyfunctional carboxylic acid and by subsequently subjecting obtained reaction product to aminolysis with at least one alkanolamine.

3. A novel β-hydroxyamide according to claim 1 or 2 characterised in, that R¹ is a group derived from at least one compound having at least two hydroxyl groups.

4. A novel β-hydroxyamide according to claim 1 or 2 characterised in, that R² is a group derived from at least one compound having at least two carboxyl groups or from an anhydride, a halide or an alkyl ester or ether of a compound having said at least two carboxyl groups.

5. A novel β-hydroxyamide according to claim 1 or 2 characterised in, that R³ is a group derived from at least one alkanolamine.

6. A novel β-hydroxyamide according to any of the claims 1-5 characterised in, that said alkyl comprises at least one ester and/or ether group.

7. A novel β-hydroxyamide according to any of the claims 1-6 characterised in, that said alkyl is linear or branched alkanyl or alkenyl.

8. A novel β-hydroxyamide according to claim 7 characterised in, that said alkanyl has 1-24 carbon atoms.

9. A novel β-hydroxyamide according to claim 7 characterised in, that said alkenyl has 2-24 carbon atoms.

10. A novel β-hydroxyamide according to any of the claims 1-6 characterised in, that said N-alkyl is N-alkanyl having 2-20 carbon atoms and that said N-cycloalkyl is cycloalkanyl having 3-20 carbon atoms.

11. A novel β-hydroxyamide according to any of the claims 1-6 characterised in, that alkoxy in said alkoxyalkyl is —(CᵣH₂ᵣO)ₚ— wherein r and p are independent integers being at least 1.

12. A novel β-hydroxyamide according to any of the claims 1-11 characterised in, that alkoxy in said alkoxyalkyl is derived from at least one alkylene oxide.

13. A novel β-hydroxyamide according to claim 12 characterised in, that said alkylene oxide is ethylene oxide, propylene oxide and/or butylene oxide.

14. A novel β-hydroxyamide according to any of the claims 1-13 characterised in, that R³ is a group of formula 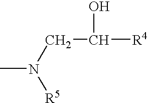
wherein R⁴ is alkyl and R⁵ is hydrogen or a group of formula 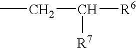
wherein R⁶ is alkyl and R⁷ is hydrogen, hydroxyl, alkyl or hydroxyalkyl.

15. A novel β-hydroxyamide according to claim 14 characterised in, that said alkyl is linear or branched alkanyl having 1-20 carbon atoms.

16. A novel β-hydroxyamide according to any of the claims 1-15 characterised in, that said compound having said at least two hydroxyl groups is a di, tri or polyalcohol of formula 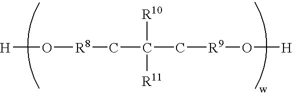
wherein w is an integer and at least 1, R⁸ is a group of formula (H₂ₓ₊₁Cₓ)_{y}—, (Oₚ₋₁H₂ᵣCᵣ)ₚ— or (Oₚ₋₁H₂ᵣCᵣ)ₚ(H₂ₓ₊₁Cₓ)_{y}—, R⁹ is a group of formula —(CₓH₂ₓ₊₁)_{y}, —(CᵣH₂ᵣOₚ₋₁)ₚ or —(CₓH₂ₓ₊₁)_{y}(CᵣH₂ᵣOₚ₋₁)ₚ and R¹⁰ and R¹¹ independently are —H, —OH or a group of formula —(CₓH₂ₓ₊₁)_{y}, —(CₓH₂ₓ)_{y}OH, —(CᵣH₂ᵣO)ₚH, —(CₓH₂ₓ₊₁)_{y}(CᵣH₂ᵣO)ₚH, wherein x, y, r and p are independent intergers being at least 1.

17. A novel β-hydroxyamide according to claim 16 characterised in, that said di, tri or polyalcohol is selected from the group consisting of a 2-alkyl-1,3-propanediol, a 2,2-dialkyl-1,3-propanediol, a 2-hydroxy-1,3-propanediol, a 2,2-dihydroxy-1,3-propanediol, a 2-hydroxy-2-alkyl-1,3-propanediol, a 2-hydroxyalkyl-2-alkyl-1,3-propanediol, a 2,2-di(hydroxyalkyl)-1,3-propanediol, a 2-hydroxyalkoxy-2-alkyl-1,3-propanediol, a 2,2-di(hydroxyalkoxy)-1,3-propanediol, a 2-hydroxyalkoxyalkyl-2-alkyl-1,3-propanediol, a 2,2-di(hydroxyalkoxyalkyl)-1,3-propanediol and a dimer, trimer or polymer of a said 1,3-propanediol.

18. A novel β-hydroxyamide according to claim 16 or 17 characterised in, that said alkyl is linear or branched alkanyl having 1-18 carbon atoms or linear or branched alkenyl having 3-18 carbon atoms.

19. A novel β-hydroxyamide according to claim 17 characterised in, that said alkoxy and/or said hydroxyalkoxy is derived from at least one alkylene oxide.

20. A novel β-hydroxyamide according to claim 19 characterised in, that said alkylene oxide is ethylene oxide, propylene oxide and/or butylene oxide.

21. A novel β-hydroxyamide according to any of the claims 16-20 characterised in, that said di, tri or polyalcohol is 2-methyl-1,3-propanediol, 2-methyl-2-ethyl-1,3-propanediol, 2-ethyl-2-butyl-1,3-propanediol, neopentyl glycol, dimethylpropane, trimethylolethane, trimethylolpropane, di(trimethylolethane), di(trimethylolpropane), pentaerythritol or dipentaerythritol.

22. A novel β-hydroxyamide according to any of the claims 1-15 characterised in, that said compound having said at least two hydroxyl groups is a monoallyl or mono(methallyl) ether of glycerol, trimethylolethane, trimethylolpropane, di(trimethylolethane), di(trimethylolpropane) or pentaerythritol.

23. A novel β-hydroxyamide according to any of the claims 1-15 characterised in, that said compound having said at least two hydroxyl groups is a diallyl or di(methallyl) ether of di(trimethylolethane), di(trimethylolpropane) or pentaerythritol.

24. A novel β-hydroxyamide according to any of the claims 1-15 characterised in, that said compound having said at least two hydroxyl groups is glycerol, diglycerol, anhydroenneaheptitol, sorbitol, mannitol.

25. A novel β-hydroxyamide according to any of the claims 1-15 characterised in, that said compound having said at least two hydroxyl groups is di, tri, or poly(hydroxy)carboxylic acid, wherein the carboxyl group or groups is/are protected.

26. A novel β-hydroxyamide according to claim 25 characterised in, that said di, tri, or poly(hydroxy)carboxylic acid is 2,2-dimethylolpropionic acid, α,α-bis(hydroxymethyl)butyric acid, α,α,α-tris(hydroxymethyl)acetic acid, α,α-bis(hydroxymethyl)valeric acid, α,α-bis(hydroxy)propionic acid, 3,5-di(hydroxy)benzoic acid, α,β-di(hydroxy)propionic acid, heptonic acid, citric acid, tartaric acid, di(hydroxy)maloic acid and/or gluconic acid.

27. A novel β-hydroxyamide according to any of the claims 1-26 characterised in, that R² is derived from a di, tri or polyfunctional carboxylic acid, from an anhydride of a di, tri or polyfunctional carboxylic acid or from a di, tri or polyalkyl ester of a di, tri or polyfunctional carboxylic acid.

28. A novel β-hydroxyamide according to claim 27 characterised in, that said alkyl is C₁-C₁₈ such as C₁-C₈ or C₁-C₄, linear or branched alkanyl.

29. A novel β-hydroxyamide according to claim 27 or 28 characterised in, that said di, tri or polyfunctional carboxylic acid is selected from the group consisting of adipic acid, azelaic acid, fumaric acid, maleic acid, phthalic acid, isophthalic acid, terephthalic acid, tetrahydrophthalic acid, hexahydrophthalic acid, succinic acid, sebacic acid, diglycolic acid, trimelletic acid, citric acid and pyromelletic acid.

30. A novel β-hydroxyamide according to any of the claims 1-29 characterised in, that said alkanolamine is selected from the group consisting of monoethanolamine, diethanolamine, mono-n-propanolamine, di-n-propanolamine, monoisopropanolamine, diisopropanolamine, mono-n-butanolamine, di-n-butanolamine, monoisobutanolamine, diisobutanolamine, mono-sec-butanolamine, di-sec-butanolamine, methylethanolamine, n-butylethanolamine, isobutylethanolamine, N-acetylethanolamine, 2-aminocyclohexanol, 2-aminocyclopentanol, 2-amino-2-methyl-1,3-propanediol, 2-amino-2-ethyl-1,3-propanediol and 2-amino-2-hydroxymethyl-1,3-propanediol.

31. A process for synthesis of a β-hydroxyamide according to any of the claims 1-30 characterised in, that said process comprises the steps of
i) subjecting a di, tri or polyalcohol of formula 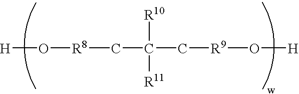
wherein w is an integer and at least 1, R⁸ being a group of formula (H₂ₓ₊₁Cₓ)_{y}—, (Oₚ₋₁H₂ᵣCᵣ)ₚ— or (Oₚ₋₁H₂ᵣCᵣ)ₚ(Hₓ₊₁C₂ₓ)_{y}—, R⁹ being a group of formula —(CₓH₂ₓ₊₁)_{y}, —(CᵣH₂ᵣOₚ₋₁)ₚ or —(CₓH₂ₓ₊₁)_{y}(CᵣH₂ᵣOₚ₋₁)ₚ and R¹⁰ and R¹¹ independently being —H, —OH, —COOH or a group of formula —(CₓH₂ₓ₊₁)_{y}, —(CₓH₂ₓ)_{y}OH, —(CₓH₂ₓ)_{y}COOH, —(CᵣH₂ᵣO)ₚH, —(CₓH₂ₓ₊₁)_{y}(CᵣH₂ᵣO)ₚH, wherein x, y, r and p are independent integers being at least 1,
to alcoholysis with a di, tri or polyalkyl ester of a di, tri or polyfunctional carboxylic acid, said di, tri or polyalkyl ester having a formula of 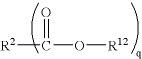
wherein R² is alkyl, aryl, alkylaryl or arylalkyl, wherein alkyl is linear or branched alkanyl having 1-24 carbon atoms or linear or branched alkenyl having 2-24 carbon atoms, R¹² is C₁-C₈ alkyl, and q is an interger and at least 2,
while removing by-product R¹²OH, and
ii) subjecting in Step (i) obtained reaction product to aminolysis with at least one alkanolamine, while removing by-product R¹²OH.

32. A process according to claim 31 characterised in, that R¹² is methyl, ethyl, propyl or butyl.

33. A process according to claim 31 or 32 characterised in, that said di, tri or polyalcohol is selected from the group consisting of 2-alkyl-1,3-propanediol, a 2,2-dialkyl-1,3-propanediol, a 2-hydroxy-1,3-propanediol, a 2,2-dihydroxy-1,3-propanediol, a 2-hydroxy-2-alkyl-1,3-propanediol, a 2-hydroxyalkyl-2-alkyl-1,3-propanediol, a 2,2-di(hydroxyalkyl)-1,3-propanediol, a 2-hydroxyalkoxy-2-alkyl-1,3-propanediol, a 2,2-di(hydroxyalkoxy)-1,3-propanediol, a 2-hydroxyalkoxyalkyl-2-alkyl-1,3-propanediol, a 2,2-di(hydroxyalkoxyalkyl)-1,3-propanediol and a dimer, trimer or polymer of a said 1,3-propanediol.

34. A process according to claim 33 characterised in, that said alkyl is linear or branched alkanyl having 1-18 carbon atoms or linear or branched alkenyl having 3-18 carbon atoms.

35. A process according to claim 33 characterised in, that said alkoxy and/or said hydroxyalkoxy is derived from at least one alkylene oxide.

36. A process according to claim 35 characterised in, that said alkylene oxide is ethylene oxide, propylene oxide and/or butylene oxide.

37. A process according to any of the claims 31-36 characterised in, that said di, tri or polyalcohol is 2-methyl-1,3-propanediol, 2-methyl-2-ethyl-1,3-propanediol, 2-ethyl-2-butyl-1,3-propanediol, neopentyl glycol, dimethylpropane, trimethylolethane, trimethylolpropane, di(trimethylolethane), di(trimethylolpropane), pentaerythritol or dipentaerythritol.

38. A process according to claim 37 characterised in, that said di, tri or polyalcohol is a di, tri or poly(hydroxy)carboxylic acid, wherein the carboxyl group or groups is/are protected.

39. A process according to claim 38 characterised in, that said di, tri, or poly(hydroxy)carboxylic acid is 2,2-dimethylolpropionic acid, α,α-bis(hydroxymethyl)butyric acid, α,α,α-tris(hydroxymethyl)acetic acid, α,α-bis(hydroxymethyl)valeric acid, α,α-bis(hydroxy)propionic acid, 3,5-di(hydroxy)benzoic acid, α,β-di(hydroxy)propionic acid, heptonic acid, citric acid, tartaric acid, di(hydroxy)maloic acid and/or gluconic acid.

40. A process according to any of the claims 31-39 characterised in, that said di, tri or polyfunctional carboxylic acid is selected from the group consisting of adipic acid, azelaic acid, fumaric acid, maleic acid, phthalic acid, isophthalic acid, terephthalic acid, tetrahydrophthalic acid, hexahydrophthalic acid, succinic acid, sebacic acid, diglycolic acid, trimelletic acid, citric acid and pyromelletic acid.

41. A process according to any of the claims 31-40 characterised in, that said alkanolamine is selected from the group consisting of monoethanolamine, diethanolamine, mono-n-propanolamine, di-n-propanolamine, monoisopropanolamine, diisopropanolamine, mono-n-butanolamine, di-n-butanolamine, monoisobutanolamine, diisobutanolamine, mono-sec-butanolamine, di-sec-butanolamine, methylethanolamine, n-butylethanolamine, isobutylethanolamine, N-acetylethanolamine, 2-aminocyclo-hexanol, 2-aminocyclopentanol, 2-amino-2-methyl-1,3-propanediol, 2-amino-2-ethyl-1,3-propanediol and 2-amino-2-hydroxymethyl-1,3-propanediol.

42. A process according to any of the claims 31-41 characterised in, that said alcoholysis is performed at a temperature of 150-250° C., such as 160-220° C.

43. A process according to any of the claims 31-43 characterised in, that said aminolysis is performed at a temperature of 150-250° C., such as 180-220° C.
